(19) United States
(12) Patent Application Publication
(10) Pub. No.: US 2025/0367017 A1
(43) Pub. Date: Dec. 4, 2025

(54) SAPHENOUS VEIN COMPRESSION SYSTEMS AND METHODS OF USE

(71) Applicant: Sun Scientific Inc., Dobbs Ferry, NY (US)

(72) Inventors: Sundaram Ravikumar, Braircliff Manor, NY (US); Vikram Ravikumar, New York, NY (US); Guy Osborne, Trumbull, CT (US); Harry Allan Alward, Shelton, CT (US); David Museau, Dobbs Ferry, NY (US)

(73) Assignee: Sun Scientific Inc., Dobbs Ferry, NY (US)

(21) Appl. No.: 18/858,327

(22) PCT Filed: Apr. 20, 2023
(86) PCT No.: PCT/US2023/019305

§ 371 Date: Oct. 20, 2024

Related U.S. Application Data
(63) Continuation-in-part of application No. 17/608,323, filed on Nov. 2, 2021.
(63) Continuation-in-part of application No. 16/846,211, filed on Apr. 10, 2020, now abandoned.

(51) Int. Cl. A61F 5/34; A61F 13/00; A61F 13/53

(52) U.S. Cl. CPC A61F 5/34; A61F 13/53; A61F 2013/00655; A61F 2013/530649

(57) ABSTRACT

A saphenous vein compression system(s) and methods of use including a wound bolster applied over the saphenous vein, a compression liner or stocking applied over the wound bolster, and a thigh therapeutic compression garment applied over both with the thigh therapeutic compression garment inflated for compression therapy on the thigh and saphenous vein of a user.

### RELATED APPLICATIONS

The present application claims priority from PCT application Serial No. PCT/US23/019305 filed Apr. 20, 2023 and entitled “Saphenous Vein Compression Systems and Methods of Use” claiming priority from provisional application Ser. No. 63/333,446 filed Apr. 21, 2022 and entitled “Saphenous Vein Compression System and Methods of Use”, and as a continuation-in-part from U.S. application Ser. No. 17/608,323 filed Nov. 2, 2021 and entitled “Therapeutic Compression System and Methods of Use” based on application Serial No. PCT/US2020/31227 filed May 2, 2020, based on application Ser. No. 62/842,165 filed May 2, 2019, and as a continuation-in-part from U.S. application Ser. No. 16/846,211 filed Apr. 10, 2020 entitled “Thigh Therapeutic Compression Apparatus, System and Methods of Use” based on provisional Ser. No. 62/378,581 filed Apr. 10, 2019, the entire contents of which are hereby incorporated herein by reference for all purposes.

### BACKGROUND OF THE INVENTION

### I. Field of the Invention

The subject invention is directed generally to a system and device for applying compression to a limb, and more particularly, to a therapeutic apparatus for applying compression to the leg of an individual in conjunction with post-ablation such as sclerotherapy as well as the treatment of conditions such as deep vein thrombosis (DVT), chronic venous insufficiency (CVI) and lymphedema, and more specifically to a saphenous vein location for compression such as post-surgery such as sclerotherapy of any other treatment for the above on a thigh. The system includes a wound dressing such as a pre-filled air bladder (“PFAB”), a compression sleeve, and a thigh therapeutic compression garment or apparatus.

### II. Background of the Related Art

Normally, a healthy leg muscle, for example, squeezes the deep veins of the legs and feet to help move blood back to the heart. One-way valves in the deep leg veins keep blood flowing back towards the heart. However, prolonged periods of standing or sitting can cause the walls of the deep leg veins to stretch. Over time, in susceptible individuals, this can weaken the vein walls and damage the valves, causing blood to pool in the veins and increase venous blood pressure. This may result in a condition known as chronic venous insufficiency (CVI).

Treatment of CVI typically involves the use of compression stockings or medical hosiery to decrease chronic swelling. Compression stockings are elastic stockings that squeeze the veins to improve venous circulation and prevent excess blood from flowing backward. Compression stockings can also help to heal skin sores or stasis ulcers that often present in conjunction with CVI. It is also common to employ compression bandages to apply pressure to the leg. In this regard, a bandage is applied with constant tension so as to produce graduated compression with the highest pressure at the ankle. However, the technique is difficult and is often done by highly skilled caregivers.

Highly effective mechanical compression devices have also been developed for treating CVI, which are disclosed, for example. in U.S. Pat. Nos. 7,276,037 and 7,559,908, the disclosures of which are incorporated by reference herein in their entireties. These devices include a flexible wrap that carries a manually inflatable air bladder and is adapted to be securely positioned around the leg of an individual to apply localized pressure to a treatment site. The device also includes a fluid-filled wound dressing that can be applied directly to the skin for applying localized pressure and even a medicament to a venous ulcer when it is enveloped by the flexible wrap. While this device is effective for applying localized compression to the leg, it is not configured to apply localized compression to the thigh of a user or the foot to prevent swelling and further improve venous circulation to the thigh, calf, or whole leg.

Lymphedema. also known as lymphatic obstruction, is another condition of localized fluid retention and tissue swelling, and is caused by a compromised lymphatic system. Treatment for lymphedema varies depending on the severity of the edema and the degree of fibrosis of the affected limb. The most common treatments for lymphedema are manual compression lymphatic massage, compression garments or bandaging. Elastic compression garments are typically worn by persons with lymphedema on the affected limb following complete decongestive therapy to maintain edema reduction.

Compression bandaging, also called wrapping, involves the application of several layers of padding and short-stretch bandages to the involved areas. Short-stretch bandages are preferred over long-stretch bandages (such as those normally used to treat sprains), as the long-stretch bandages cannot produce the proper therapeutic tension necessary to safely reduce lymphedema and may in fact end up producing a tourniquet effect. During activity, whether exercise or daily activities, the short-stretch bandages enhance the pumping action of the lymph vessels by providing increased resistance for them to push against, thus encouraging lymphatic flow and helping to soften fluid-swollen areas.

Known methods for CVI and lymphedema treatment, like compression bandaging, have several disadvantages. The bandaging is time consuming and the effectiveness is limited to the skill of the provider. In some instances, bandages can be applied too tightly or too loosely and may slip from their intended position, decreasing their effectiveness. When this occurs, bandages must be taken off and reapplied, further increasing the time of application and decreasing the consistency of application of the therapy. Further, when the compression bandaging is on the thigh area of the leg, the bandaging slips down the leg when the user walks or moves around even in bed. possibly due to the anatomical shape of the thigh tapering towards the knee area.

The effectiveness of many of the current compression therapies is limited by the application of current products. Because current compression therapy is done either with manual wraps or electromechanical systems, they require either a skilled medical professional to apply and/or the need for the patient to be stationary for extended periods of time. Although stockings and/or bandages can be worn by patients and self-administered, they are very difficult for the patient to put on and pose a challenge for unskilled medical professionals to apply consistently and effectively. Again though, the stockings and/or bandages will slip down the thigh towards the knee once the user walks or moves around.

CVI and lymphedema may also result in DVT, which is an affliction that causes blood clots particularly in the lower extremities of the legs. When a patient is not ambulatory, the patient faces an elevated risk factor of creating a blood clot. These blood clots, which often accumulate or reside in the patient's calf or thigh, are not. in and of themselves, overly dangerous. However when the blood clot breaks loose, they create a pulmonary embolism which can get lodged in the patient's heart, brain or lungs where it can cause significant damage or death. It is estimated that in each year 2 to 2.5 million Americans are afflicted by DVT causing 600,000 patients to seek medical care with 300,000 patients succumbing to the effects of the pulmonary embolism. Thus, a system and apparatus and method of use to treat DVT is needed.

Many of the current treatment options for CVI and lymphedema cause venous ulcers including the use of current known devices, apparatus, bandages, stocking, hosiery and the like. A venous ulcer is damage and loss of skin above the ankle that is the result of a problem with the veins in the leg. Venous ulcers typically develop on either side of the lower leg, above the ankle and below the calf. They are difficult to heal and often recur. They also develop on the thigh, on the inner portion of the leg thigh or outer thigh area as well as near the groin area on the leg. Further, venous ulcers may develop around the knee, behind the knee, along the sides and on the front of the knee as well. It is estimated that in the US alone about 3 million people per year develop pressure ulcers which left untreated can cause major complications, infections, pain, and the like.

The veins of the leg are divided into the superficial and deep systems according to their position relative to the fascia. The deep veins, which come together to form the popliteal and femoral veins lie within the fascia and are responsible for the venous return from the leg muscles. Dilated valveless sinusoids also lie within the fascia (more particularly in the soleus and gastrocnemius muscles). The sinusoids fill with blood when the leg is at rest.

The long saphenous vein which runs along the medial side of the leg from foot to groin and the short saphenous vein which runs at the back of the calf from foot to knee are the major vessels of the superficial venous system. These vessels lie outside the fascia and are responsible for the venous return from the skin and subcutaneous fat. Compression on the long saphenous vein may be recommended in treatment of certain conditions whereas in other conditions there may be less compression recommendation on the long saphenous vein on the medial side of the leg and more compression recommended on the short saphenous vein of the leg which runs on the back portion of the leg, including the thigh. Further, post-ablation surgery such as sclerotherapy, the saphenous vein should be compressed sufficiently to promote healing and reduce complications.

Communicating veins, sometimes called perforators because they perforate the deep fascia, join the two systems. The perforators, like the other veins in the leg, contain valves that permit the flow of blood in one direction only, from the outer or superficial system inwards to the deep veins.

The venous pressure at the ankle of a subject who is lying supine is around 10 mm Hg, but on standing this will rise considerably due to an increase in hydrostatic pressure (equivalent to the weight of a vertical column of blood stretching from the point of measurement to the right auricle of the heart).

During walking, as the foot is dorsally flexed, the contraction of the calf muscle compresses the deep veins and soleal sinuses thereby emptying them of blood. As the foot is plantarly flexed, the pressure in the veins falls, the proximal valves close, and the veins are refilled by blood passing through the perforators from the superficial system. During this cycle, in a normal leg, the distal valves of the deep veins and the valves of the perforators will ensure that the expelled blood can go in only one direction—upwards, back to the heart.

Blockage or damage to the venous system will cause disruption to normal blood flow. which may manifest itself in a number of different ways according to the site and extent of the damage. If the valves in the superficial system are affected, venous return will be impaired and blood may accumulate in the veins causing them to become distended, leading to the formation of varicosities (varicose veins). Such varicosities may be located in the thigh, knee, calf, ankle or foot area of the user's leg.

If the function of the perforator valves is impaired, the action of the calf muscle pump will tend to cause blood to flow in the reverse direction into the superficial system increasing the possibility of damage to the superficial vessels. There may be minimal blood flow upward into the thigh and the rest of the body, causing issues for the patient along the body including the thigh, calf, ankle and entire leg of the patient.

Following a deep vein thrombosis that results in complete or partial obstruction of a deep vein, the unrelieved pressure produced by the calf muscle pump on the perforator valves may cause these to become incompetent. If this occurs, there will be a large rise in the pressure in the superficial system, which may force proteins and red cells out of the capillaries and into the surrounding tissue. Here, the red cells break down releasing a red pigment that causes staining of the skin, an early indicator of possible ulcer formation. The ulcer formation can be in any part of the body, including the leg, groin, thigh, knee, calf, ankle and foot.

Venous leg ulcers are generally shallow and red in color. The skin surrounding the ulcer is frequently discolored due to the staining described previously. Incompetent perforating vein valves can also cause malleolar venules to become dilated and appear as fine red threads around the ankle as well as the thigh, knee, calf or foot are of the leg or any area of the leg. This condition, called ankle flair, is also diagnostic of a venous ulcer. The condition may also be seen on the thigh, including the inner medial thigh area or outer lateral thigh area, or around the knee or groin area of the patient's body.

Arteries transport oxygen replenished blood from the heart to the rest of the body. Veins return oxygen depleted blood back to the heart. When the veins in the lower extremities of the body have difficulty transporting blood back to the heart, a condition develops called chronic venous insufficiency (CVI), also known as chronic venous disease (CVD). CVI most commonly occurs as the result of a blood clot in the deep veins of the legs, a disease known as deep vein thrombosis (DVT). CVI also results from pelvic tumors and vascular malformations, and sometimes occurs for unknown reasons. When a person is standing or sitting, blood in the veins of the legs flows in an upward direction. When the person walks, the calf muscles and muscles in the feet contract to squeeze the veins and push the blood upward. To keep the blood flowing upward and prevent it from flowing downward. the veins contain one-way valves. CVI occurs when these valves become damaged and allow the blood to leak back downward in the opposite direction. Such valve damage may occur as the result of aging, extended sitting or standing, or a combination of aging and reduced mobility. When the veins and valves become weakened and the blood does not properly flow up to the heart, blood pressure in the veins of the lower extremities can stay elevated for long periods of time, leading to CVI. This condition is more common in older individuals, and if not properly treated, can lead to burst capillaries, local tissue inflammation, internal tissue damage, varicose veins, ulcers, and open sores on the skin's surface. The burst capillaries can be seen on the patient's leg including the thigh, knee and groin.

CVI can diminish the capacity of the venous system and increase the workload of the lymphatic system in the affected area. The lymphatic system must then transport larger volumes of water and protein to reduce the fluid load in the affected tissues of the legs, a situation which is especially difficult for patients with lymphedema, varicose veins, and other lower extremity pathology.

One non-surgical option often used to help prevent or treat the leg extremity pathologies discussed above is the use of compression stockings. Compression stockings help prevent leg fatigue, ankle and foot swelling, spider veins, and varicose veins. They improve circulation in the legs, especially when used in conjunction with frequent exercise and leg elevation. Compression stockings maintain pressure on the legs while allowing for normal ambulation. Increasing pressure in the tissues beneath the skin reduces excess leakage of fluid from the capillaries and increases absorption of tissue fluid by the capillaries and lymphatic vessels. In addition, the increased pressure decreases the size of the veins, which causes the blood to flow faster and helps prevent it from pooling.

Compression stocking tightness typically varies between 15-50 mm HG. The tightness of a given stocking depends on its particular configuration and class. For example, stockings having a compression pressure of 15-20 mm HG are considered light compression stockings. Class1 stockings are 20-30 mmHg, class II stockings are 30-40 mm Hg. and class III stockings are 40-50 mmHg. While such compression stockings are a commonly utilized non-invasive treatment of leg pathology. the issues they present are numerous. Wearing a tightly fitting stocking can be tedious or time consuming to put on. and may require help from another person if the wearer is injured. elderly. or has some form of disability. Any tight-fitting stocking to be worn on the thigh area has a harder time being put on as the user has to pull the tight fitting stocking over the foot. ankle. calf, knee and finally up to the thigh area. Again, due to the shape of the thigh, these tight-fitting stocking will slip down the thigh at some point due to the user walking or moving about.

In addition, the pressure applied by the stocking generally stays relatively constant during use without any option of increasing or decreasing the tightness level. As compression stockings are repeatedly worn, they lose elasticity and thus tightness over time. Once such prescribed elasticity and tightness is lost. the stocking is of little or no value, and needs to be replaced on account of its looseness, which requires buying a new pair to obtain the desired pressure. Further, given the shape of the thigh, the top or upper portion of the stocking must be very tight in order to prevent slippage down the thigh, which can in tum lead to complications of its own due to the excessive tightness.

Medical hosiery represents a useful and convenient method of applying compression to normal shaped legs in order to prevent the development or recurrence of leg ulcers. However, these stockings are of limited value in the treatment of active ulceration, being difficult to apply over dressings. In such situations compression bandages currently represent the treatment of choice. Compression bandages apply a pressure to the limb that is directly proportional to bandage tension but inversely proportional to the radius of curvature of the limb to which it is applied. This means, therefore, that a bandage applied with constant tension to a limb of normal proportions will automatically produce graduated compression with the highest pressure at the knee. This pressure will gradually reduce up the thigh as the circumference increases.

As can be readily appreciated, it is cumbersome and difficult to apply uniform tension to the compression bandage as it is applied to the treated limb, and thus this is accomplished only by highly skilled caregivers. Moreover, once secured to the treated limb, care and attention must be given to ensure that the bandage does not slip or become displaced as this will lead to multiple layers fanning, which in tum may lead to localized areas of high pressure, which can place the patient in direct risk of skin necrosis.

Mechanical compression treatments have also been proposed. An exemplary compression device is described in U.S. Pat. No. 5,031,604 to Dye. As generally described at col. 2, lines 33 et seq. an arrangement of chambers are provided that circumscribe the leg. An active pneumatic control system controls the pressure in the chambers to squeeze the leg near the ankle and then squeeze sequentially upward toward the knee in order to move blood from the extremity toward the heart. As noted in col. 4, lines 20-59 of U.S. Pat. No. 6,488,643 to Tumey et al., the mechanically produced compression levels may produce ischemic (i.e., localized tissue anemia) not noted at similar compression levels obtained through bandaging. It may also produce cuffing (i.e., a reduction in leg pulsatile blood flow). The pneumatic control system is also bulky and heavy, which severely limits the mobility of the patient during treatment. Moreover, the pneumatic control system fails to provide a mechanism to ensure that excessive pressure, which can cause necrosis, is not applied to the treated limb. These limitations have resulted in most mechanical compression devices being contraindicated for patients exhibiting DVT. Consequently, those skilled in the art have to date avoided such mechanical compression devices for the treatment of venous ulcers or edema of the extremities.

Co-owned U.S. Publication No. 2004/0193084, which is hereby incorporated by reference herein in its entirety, discloses a device for applying pressure to the human leg for use in conjunction with treatment of varicose veins. The device includes a flexible member and at least one air bladder chamber integral thereto that are adapted to securely wrap around the human leg. A tube in fluid communication with the air bladder chamber(s) extends to an air pumping mechanism that operates to inflate the air bladder chamber(s) to a pressurized state. The flexible member preferably includes an opening at the knee joint level to enable a patella to protrude therethrough. In addition, the flexible member preferably extends below knee joint level and is adapted to securely wrap around a lower portion of a leg to provide stability to the leg. Preferably, the air bladder chamber of the device is substantially longer in a first dimension than in a second dimension orthogonal thereto such that the air bladder chamber can be positioned to cover a portion of the human leg that is relatively long in the vertical dimension and narrow in the horizontal dimension.

Co-owned U.S. Pat. No. 7,276,037, which is hereby incorporated by reference herein in its entirety, discloses an apparatus for applying compression therapy to an extremity of the human body, such as a portion of the human leg. The device includes a flexible member and an air bladder chamber. The flexible member is adapted to wrap around the extremity to secure the air bladder chamber to the extremity. An air pumping mechanism is operated to inflate the air bladder chamber to a pressurized state. One or more fluid-filled pressurized members are provided, each separate and distinct from the flexible member and the air bladder chamber and thus readily moveable relative to the flexible member and the air bladder chamber. The pressurized member(s) is operably disposed between the extremity and the flexible member whereby it applies increased localized pressure to the extremity during use. Preferably, the air bladder chamber is substantially longer in a first dimension than in a second dimension orthogonal thereto such that it can extend longitudinally along the extremity to cover a relatively long and narrow portion of the extremity. The position of the air chamber can be readily adapted to apply local pressure to desired body parts (such as a certain venous channel). The pressurized member(s) can be positioned during use such that it covers a venous ulcer (or other treatment sites) and applies increased localized pressure to the treatment site in order to promote healing.

Surgical treatments for leg vascular issues include Sclerotherapy which is a medical procedure used to eliminate varicose veins and spider veins. Sclerotherapy typically involves an injection of a solution (generally a salt solution) directly into the vein. The solution irritates the lining of the blood vessel, causing it to collapse and stick together and the blood to clot. Post-operative procedures may require compression bandaging or stockings, both of which have the problems described above in regard to slippage down the thigh to the knee as well as cost and need for a skilled level of knowledges in order to properly bandage the sclerotherapy treatment area. A need exists for a compression system along the saphenous vein which the user can apply themselves without a caregiver or medical professional. A need exists for a saphenous vein compression system with multiple layers of compression to assist in healing and reduce pain, complications, risk of infection and the like.

Compression is standard treatment for venous disease. Most venous disease is related to venous-hypertension secondary to incompetent vein valves and compression helps reduce the available space in the venous column and improve venous return, both of which reduce venous hypertension. In treating venous leg ulcers and varicose vein treatments, the leg/thigh is compressed in general to achieve specific venous compression. Unfortunately, the intended site and level of compression is sometimes not adequately achieved with standard compression products and techniques. For example, with varicose vein treatments of the thigh, it is often seen that the specific vein is not adequately compressed due to the size of the limb and depth of the vein. In both cases, a “bolster” which builds up the specific spot increases local compression at the area where the bolster is placing and increases mechanical stress at the site as well while also off-loading stress on the entire arca. This leads to less complications, quicker recovery time and improved outcomes.

Thus a need exists for a simple saphenous vein compression system including a wound dressing for venous disease treatments to improve and standardize localized bolstering. A need exists for a dressing which includes a standardized bolster height as well as the ability to distribute the pressure evenly across the underlying anatomy resulting in optimal compression and comfort.

A further need exists for a saphenous vein compression system in which a bolster dressing is combined with a compression garment or compression dressing placed on a saphenous vein location on the inner thigh of a patient or user.

Further a need exists for a system in which a multiple of compression garments have separate active pressures. For instance, the inventive thigh compression system could be used in conjunction with a lower leg compression apparatus such as that described (and incorporated herein as reference) in U.S. Pat. No. 9,033,906 and U.S. Pat. No. 7,967,766 and U.S. Pat. No. 7,559,908 and U.S. Ser. No. 13/444,600 and U.S. Ser. No. 16/846,211 and therefore the user could wear two separate therapeutic compression apparatus on the entire leg and could also include an optional knee wrap. In this instance the user could have one pressure level on the inventive thigh therapeutic compression system whereas a calf compression garment could have a second pressure level and both compression garments are connected to one pneumatic pump configured to have multiple pressure outlets. Such a need exists for such a system.

All current known treatment apparatus, devices, bandages, stockings and hosiery have the problems of stability (no slippage), maintaining sufficient effective pressure without overpressure complications, maintaining compression and the like. Further all known apparatus, devices, bandages, stockings and hosiery, though especially the current treatment apparatus and devices, are only capable of connecting to one source of compression or inflation means and no universal inflation port of connector is known—wherein a patient could vary treatment through varying the inflation source and inflation means for the treatment apparatus or device.

Other known problems with the current treatment apparatus and devices, bandages, stockings and hosiery is the requirement that a skilled caregiver apply the current treatment apparatus and devices, bandages, stockings and hosiery. Such a skilled caregiver may not be available to all patients, notably those without long-term care insurance or provided a skilled home health aid. Yet another known problem is leakage of set compression within the treatment apparatus, and devices, bandages. stockings and hosiery resulting in an ineffective treatment and ineffective apparatus or device and the like which may be rendered useless to the patient and user. A further problem with the current treatment apparatus and devices, bandages, stockings and hosiery is that the inflation means or source of compression is set up as either manual or mechanical or electrical and cannot be interchanged in that the inflation port or inflation means not universal and interchangeable. Yet another problem with the current treatment apparatus, devices, bandages, stockings and hosiery is that the inflation means or source is either static intermittent and again cannot be changed during the treatment with such apparatus or device. Yet another problem with the current treatment apparatus and devices, bandages, stockings hosiery is that the apparatus and device, bandages, stocking and hosiery slips down the leg gravity and/or walking or movement of the user. Further, the bandages and dressings can re or down then applying pressure to the leg which is not wanted resulting in circulation problems, and lack of blood flow to other parts of the leg due to the vein or artery being compressed to hard and blocking blood flow.

The system, apparatuses, assemblies and methods of the subject invention provide benefits and advantages that may overcome a number of problems with respect to known compression technologies, particularly the problems that arise due to the difficulty of apply current compression wrap technologies on a saphenous vein or any vein such as post-ablation treatment or sclerotherapy. The subject invention provides an alternative to known technologies that employ tight-fitting therapeutic elastic garments, which cause patients discomfort, and lose their elasticity as well as have slippage down the leg and thigh when moving even remotely from their bed or couch to the bathroom, and therefore negatively reduces their effectiveness over time. Those skilled in the art will readily appreciate that it would be beneficial to provide a therapeutic compression device and system for treating CVI, DVI, lymphedema that is adapted and configured to apply localized compression to the inner thigh well as the leg, thigh, calf and/or foot to prevent swelling and further improve venous circulation that may also be self-administered by a patient effectively.

Without limitation, the inventive saphenous vein compression system may be used with any required compression therapy such as venous disease, vascular disease, lymphedema and the like. The inventive saphenous vein compression system may be used to treat any general swelling as well as being used post operatively, for example including in cases of sclerotherapy vein ablation. The inventive thigh therapeutic compression apparatus may be used by a person for compression therapy such as athletes and lactic acid build up, or pregnant women, as well as any individual who walks a lot or is on their feet at work for period of time. The inventive saphenous vein compression system may be used to compress the long saphenous vein which runs along the medial side of the leg from foot to groin and the short saphenous vein which runs at the back of the calf from foot to knee are the major vessels of the superficial venous system. The inventive saphenous vein compression system may also be used to compress the vessels which lie outside the fascia and are responsible for the venous return from the skin and subcutaneous fat. The inventive saphenous vein compression system may be used in treatment where there may be different compression profiles on the medial long saphenous vein compared to a different compression profile on the short saphenous vein on the back of the thigh, or any combination. Other uses for the inventive saphenous vein compression system may be envisioned.

### SUMMARY OF THE INVENTION

The subject invention is directed to a therapeutic compression system and methods of use. The saphenous vein compression system has a bolster wound dressing and a compression garment either alone with a compression housing or elastic liner under the garment and above the bolster dressing, with the bolster dressing configured for positioning along the saphenous vein and then covered with the elastic liner and the thigh therapeutic compression garment apparatus. The system may be placed on the inner thigh or upper leg of the patient or user. The saphenous vein compression system may include a stabilization means to stabilize the compression garment from slipping down the thigh of the user when in use.

The saphenous vein compression system may comprise at least one pre-filled air bladder dressing operatively associated with the saphenous vein for applying pressure to a treatment site on the limb such as a leg or the saphenous vein location. The saphenous vein compression system may also include a thigh therapeutic compression apparatus such as those disclosed in U.S. Ser. No. 16/846,211 which is incorporated herein.

In another embodiment of the present invention, the saphenous vein compression system includes a thigh therapeutic compression garment that has two bladders, a lateral bladder and a medial bladder. The medial bladder has a gradient compression profile based on the geometric location of the spot welds located on the medial bladder. The lateral bladder may be connected to the medial bladder and the lateral bladder also has a separate gradient compression profile based on the geometric location of the spot welds on the medial bladder. In another embodiment there is no lateral bladder and only a medial bladder. In yet another embodiment the lateral bladder is not connected to the medial bladder as the medial bladder is closed off and each bladder is inflated via a separate inflation port, and each of the lateral bladder and medial bladder may have a different gradient compression profile based on the predetermined configuration of the sport welds and/or lines within each of the bladders.

The securing means for the compression garment may be configured in one embodiment to be attached to a belt or other means so as to stabilize the saphenous vein compression system to the user when in use. In another embodiment the securing means is configured to have a belt inserted within the means of at least one aperture to stabilize the therapeutic compression apparatus on the user when in use. In yet another embodiment the stabilization means may be a garter belt configuration with a connecting means to a series of apertures located on the proximal end of the separate wrap. In any of these embodiments, the securing means may also be configured as an adjustable strap around the waist, or hip, or upper thigh area of a user.

The saphenous vein compression system may further comprise an adjustable belt along a proximal horizontal edge of the compression garment for securing it around the limb such as for instance the leg. The compression garment of the saphenous vein compression system may be formed at least in part of a non-clastic composite material comprising a plurality of distinct layers. In one embodiment, the composite material may comprise three distinct layers: an inner laminate layer, an outer hook-compatible layer, and a middle non-elastic layer provided between the inner and outer layers. The composite material may also be provided with a plurality of stitched darts and gathers for contouring the apparatus to the limb such as for instance the leg or thigh area of the leg.

A method of the invention includes the therapeutic treatment system used to treat CVI, DVT and/or lymphedema bas well as in use post-ablation surgery such as sclerotherapy for example, by applying the bolster dressing to the limb of a patient such as with an adhesive and then applying a compression housing or liner or stocking over the bolster dressing followed by applying a thigh therapeutic compression garment or dressing over both the elastic housing or liner and the bolster dressing. If the thigh therapeutic compression garment is an inflatable wrap, then the method includes applying the primary and secondary wraps around a limb by a patient and inserting an inflation means into an inflation port and inflating the one or multiple bladders within the primary and secondary wraps and maintaining a certain pressure or compression profile along the medial saphenous vein and/or the lateral saphenous vein, or to treat the CVI, DVT and/or lymphedema or assist in healing post-ablation surgery such as sclerotherapy or the like.

Another embodiment of the present invention includes a system assembly according to the invention including pressure mechanism having a flexible member for attachment to a limb and an air chamber which may be pumped up into a desired pressurized state, a separate relatively small pre-filled air bladder, an absorbent foam, sponge or dressing coupled to the pre-filled air bladder, and a suction conduit coupled to a source of negative pressure (suction) and in fluid communication with the absorbent foam, sponge or dressing. In a preferred embodiment, the pre-filled air bladder, the absorbent foam, sponge or dressing and the suction conduit are formed together as a unit.

According to one aspect of the invention, the flexible member of the pressure mechanism is adapted to wrap around a leg or arm and over the pre-filled air bladder in order to secure the pre-filled air bladder and the foam, sponge or dressing to a wound or ulcer in the extremity. Thus, the flexible member is provided with some fixation structure such as a hook and loop closure mechanism. An air pumping mechanism is preferably coupled to the air chamber of the pressure mechanism in order to inflate the air chamber to a pressurized state. The air chamber of the pressure mechanism is preferably designed to apply pressure or compression along a predefined area (e.g., the saphenous vein of a leg) as opposed to around an entire limb.

According to another aspect of the invention, the suction conduit is located either between the pre-filled air bladder and the absorbent foam, sponge or dressing which is adhered to the small air bladder, or the pre-filled air bladder is formed as a donut with a central opening and the suction conduit extends through the central opening. By coupling the suction conduit to a source of negative pressure, exudate from the wound or ulcer is sucked through the foam, sponge or dressing into the suction conduit.

One of the methods of the invention include locating the bolster dressing such as a pre-filled air bladder and foam, sponge or dressing over a wound or ulcer on a limb such as saphenous vein location wrapping the flexible member of the pressure mechanism around a limb with the air chamber located over the pre-filled air bladder/absorbent foam, sponge or dressing, and fastening the pneumatic mechanism in place with the fixation structure. When the apparatus is properly located and affixed, limb, the air chamber is inflated, preferably to 30-40 mm Hg, thereby applying pressure or compression to the limb and more specifically via the pre-filled air bladder to the wound. The suction apparatus is activated by turning on the source of negative pressure, and exudate from the wound or ulcer is pulled through the absorbent foam, sponge or dressing into the suction conduit.

Another embodiment of the present invention includes a thigh therapeutic compression garment or apparatus for applying intermittent pressure or compression to a portion of the human body, such as an area of the human leg, which assists with the healing and treatment of various conditions such as post-ablation surgery or treatment, venous ulcers or wounds by promoting blood flow into and out of the area and by increasing drainage. The thigh therapeutic compression garment may be a wrap including a thigh bladder or a foot bladder and a leg bladder, each having inflatable chambers that accommodate an entering fluid by inflating. The bladders are fluidly coupled by a fluid conduit, and each is preferably equipped with a means for locating it on a portion of the body. In a preferred embodiment, the thigh bladder is position between the pelvis or groin or hip area of the user and the knee of the user. As a person walks while wearing the apparatus, a portion of the thigh bladder deflates as the person's leg moves foot (heel) strikes the ground due to the external pressure placed on the foot bladder, thereby forcing fluid out of the foot bladder, through the fluid conduit, and into the leg bladder, which raises the pressure therein. As the person's foot rolls from heel to toe in the standard walking motion, the external pressure from the person's weight is removed from the foot bladder, resulting in the pressure of the leg bladder being higher than the pressure in the foot bladder. Fluid thus flows back through the fluid conduit and into the foot bladder, which then inflates again to its original state, such that the pressures of the foot bladder and leg bladder are equalized. This process repeats as a person walks, thereby creating a pumping or kneading force on the leg as the pressure in the leg bladder intermittently increases and decreases, thereby promoting blood flow, fluid drainage, treatment, and healing to various parts of the leg.

These and other aspects of the contacts of the subject invention will become more readily apparent from the following description taken in conjunction with the drawings.

### BRIEF DESCRIPTION OF THE DRAWINGS

So that those having ordinary skill in the art to which the subject invention pertains will more readily understand how to make and use the apparatuses of the subject invention. preferred embodiments thereof will be described in detail herein below with reference to the drawings. wherein:

FIG. 1 is a perspective view of one embodiment of the bolster dressing of the present invention including a thigh therapeutic for use on either leg of the user (left or right);

FIG. 2 is a top view of the embodiment of FIG. 1;

FIG. 3 is a side view of the embodiment of FIG. 1;

FIG. 4 is a view of one embodiment of the thigh therapeutic compression apparatus for the left leg of a user as part of the inventive system, the thigh therapeutic compression apparatus constructed in accordance with the present invention, including one air bladder having a medial bladder portion joined to a lateral bladder portion and including a stabilization means having a belt located at the user's waist and connected to the thigh therapeutic compression apparatus via an aperture on the proximal end of the apparatus;

FIG. 5 is an exploded view of the embodiment of FIG. 4;

FIGS. 6A-6H are an embodiment of the system and method of the present invention including a bolster dressing, an elastic liner and a compression garment on the thigh of the patient in an inflated state showing the method of placing the bolster dressing along inner portion of the user's left thigh along the saphenous vein with the adhesive and foam bottom of the bolster dressing facing upward as shown in FIG. 6A, then placing an elastic liner over the bolster dressing on the thigh of the user as shown in FIG. 6B, then locating the compression garment along the patient's thigh with the securing means around the waist as shown in FIG. 6C, the locating the compression garment along the user's back and outer thigh and saphenous vein in an open position as shown in FIG. 6D, then closing and wrapping the compression garment on the user's thigh with the bolster dressing along the inner thigh and saphenous vein of the patient as shown in FIG. 6E, then the user adjusts the therapeutic thigh compression apparatus upward on the thigh as shown in FIG. 6F, then inflating the compression garment and applying increased compression along the saphenous vein as shown in FIG. 6G, with another embodiment of the inflation port including the cap shown in FIG. 6H; and

FIG. 7 is a full lower body view of the present invention including a bolster dressing, an elastic liner and a therapeutic thigh compression garment in a closed position on the thigh of the user.

### DETAILED DESCRIPTION OF THE PREFERRED EMBODIMENTS

Preferred embodiments of the subject invention are described below with reference to the accompanying drawings, in which like reference numerals represent the same or similar elements. ordinary skill in the art would appreciate that while the apparatuses discussed herein relate to compression therapy of the leg such as the inner thigh and saphenous vein location, the scope of the invention is not limited to those exemplary applications and may be sized and shaped for the anatomical portion for which compression therapy is needed.

The subject invention provides compression to a patient's limbs, including the extremities, including for example, the inner thigh and saphenous vein location on the upper leg of a user and more specifically the thigh of a user, in a manner that is simpler and more convenient than current systems. Any limb or body part may be compressed by the instant therapeutic compression apparatus such as for instance a foot, calf, thigh, knee, leg, hip, buttocks, waist, torso, ribs, shoulder, arm, hand, fingers, neck, head or the like.

The subject invention provides system for providing compression and preventing swelling of a limb such as for instance the thigh using a bolster dressing and a thigh therapeutic compression garment to facilitate increased compression along the saphenous vein location on the patient. Optionally an elastic liner can be placed over the bolster dressing and under the compression garment. The inventive saphenous vein compression system creates compression in a manner that allows for consistent and increased compression to the saphenous vein area as well as safe, comfortable, convenient, effective, self-application by the patient.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges and are also encompassed within the invention. subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention. exemplary methods and materials are now described. All publications mentioned herein are incorporated herein by reference to disclose and describe the methods and/or materials in connection with which the publications are cited.

It must be noted that as used herein and in the appended claims, the singular forms “a”, “an” and “the”' include plural referents unless the context clearly dictates otherwise. Thus, for example, reference to “a stimulus” would include a plurality of such stimuli and reference to “the signal” would include reference to one or more signals and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may differ from the actual publication dates which may need to be independently confirmed.

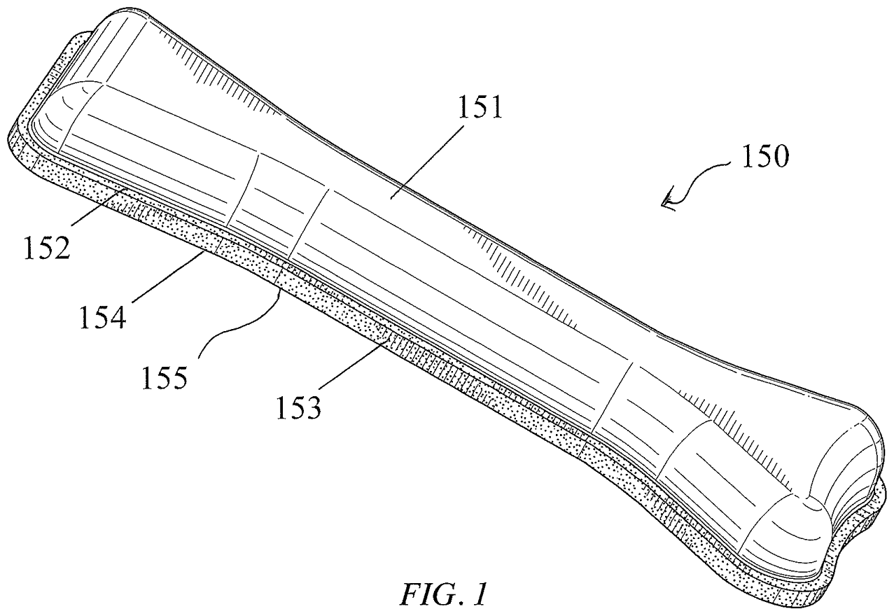

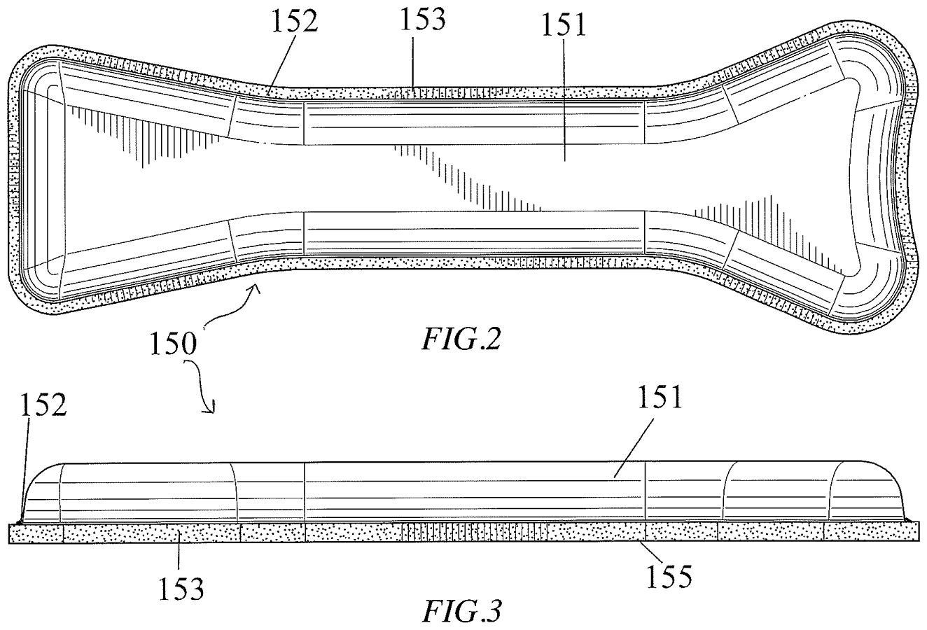

Referring now to FIG. 1, there is illustrated an exemplary embodiment of a saphenous vein compression system 100 according to the subject invention including a bolster dressing 150 and a thigh therapeutic compression garment 160 over the bolster dressing 150. The bolster dressing 150 is configured to apply localized pressure or compression along the saphenous vein of a patient when applied to the patient's skin. As shown in FIGS. 1-3, this embodiment of the bolster dressing 150 is in the shape of a dog bone so as to apply localized pressure or compression to the saphenous vein of the patient. Other shapes may be employed as well in embodiments not shown such as oval, rectangle, square, circle, crescent, “U” shape or any other known shape. In one embodiment of the bolster dressing 150 as shown in FIGS. 1-3, the bolster dressing 150 includes a pre-filled air bladder 151 configured to provide elevation, compression and bolstering, connected via a connecting means 152 to a foam portion 153 to absorb any bleeding or leakage of dye or pus or the like from an ablation or other surgical location. The foam portion 153 also includes in this example an adhesive layer 154 which is compatible to the human skin for application of the bolster dressing 150 to the patient's skin. The bolster dressing 150 may also include a paper, polymer, coating, film or other material which is a layer 155 configured to keep the adhesive inactive during shipping and storage but such layer 155 is removed just prior to application onto the skin of the patient.

The bolster dressing 150 is configured to apply localized increased pressure or compression on the target area, such as the saphenous vein on the patient post-ablation or even as an ongoing treatment for CVI, DVT and lymphedema. The bolster dressing may include a pre-filled air bladder 151 which is a set height over the entire length of the bolster dressing 150 so as to distribute the compression or pressure across the entire skin in connection with the bolster dressing 150. The foam portion 153 may be comprised of any material compatible to the human skin such as by way of example only an open cell polyurethane foam. The foam portion 153 may reduce discomfort to the patient as well as providing absorption of any bleeding or other oozing liquids on the skin of the patient. The bolster dressing is absorbent so as to assist in exudating any liquids from the wound area such as blood, pus, seepage and the like. Besides foam any absorbent material compatible with the patient or user's skin may be employed including but not limited to gauze, cotton, woven materials, lint, plasters, polymers, and various synthetic wound dressings including hydrogels, hydrocolloids, alginates, synthetic foam dressing, silicone meshes, tissue adhesives, vapor-permeable adhesive films and silver/collagen containing dressing The bolster dressing 150 also includes an adhesive layer 154 for adhesion to the patient's skin. The adhesive layer 154 may be comprised of any adhesive material compatible to the human skin, such as by way of example only a siliconized adhesive layer. The bolster dressing 150 may include a cover layer 155 to cover the adhesive layer during shipping and storage such as a paper or polymer or film which is removed just prior to application of the bolster dressing 150 to the patient's skin.

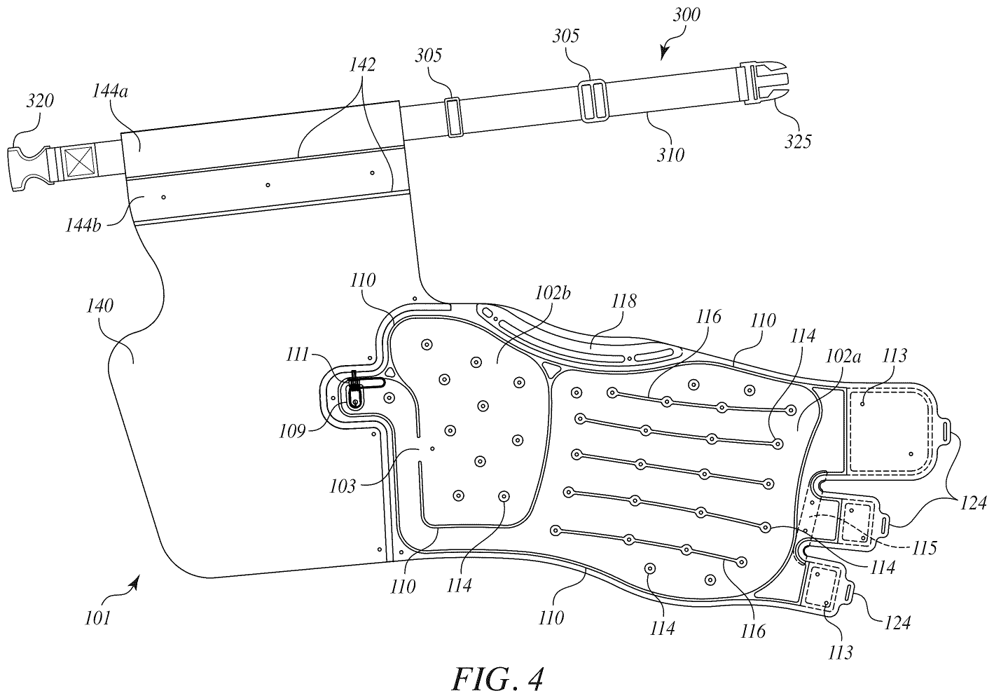

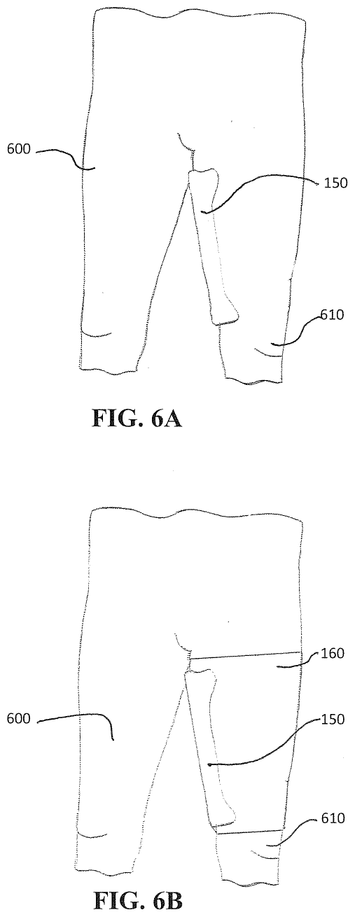

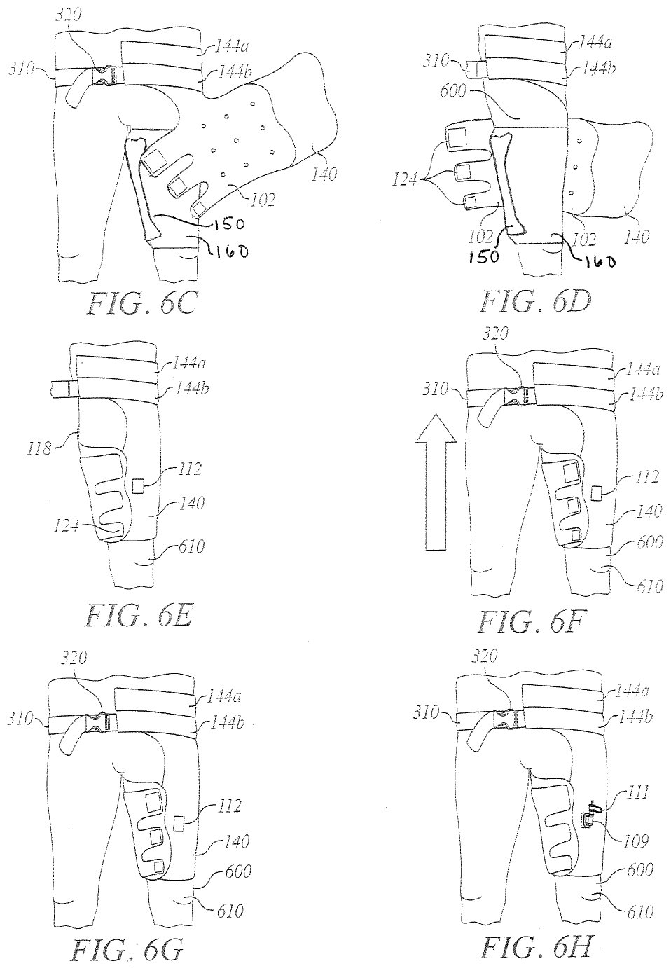

The inventive saphenous vein compression system also includes a compression liner or sleeve or stocking or housing 160 which is placed over the bolster dressing 150. The compression liner or sleeve or stocking or housing 160 may be any dressing or garment capable of compression to the body part such as the leg or inner thigh of the patient. Examples of compression liners or sleeves or stocking or housing 160 include but are not limited to elastic tubing, elastic housings, elastic dressings, partially elastic and inelastic dressings, compression dressings, compression bandages, compression stockings, compression sleeves, compression wraps, inflatable compression wraps, gradient compression wraps, and any compression type apparatus or device or assembly. As shown in FIGS. 3-5 and 6A-6D in one embodiment of the present invention, the compression liner or sleeve or stocking or housing garment 160 is a compression sleeve located on the user's thigh above the knee and below the hip area, but any other shape may be employed including but not limited to a full leg stocking from the foot area up to the hip, a partial leg sleeve from the ankle up to below the hip area, two leg stockings, or any other combination of coverage of one or two legs and up the waist area, as long as the saphenous vein is covered over the applied bolster dressing 150.

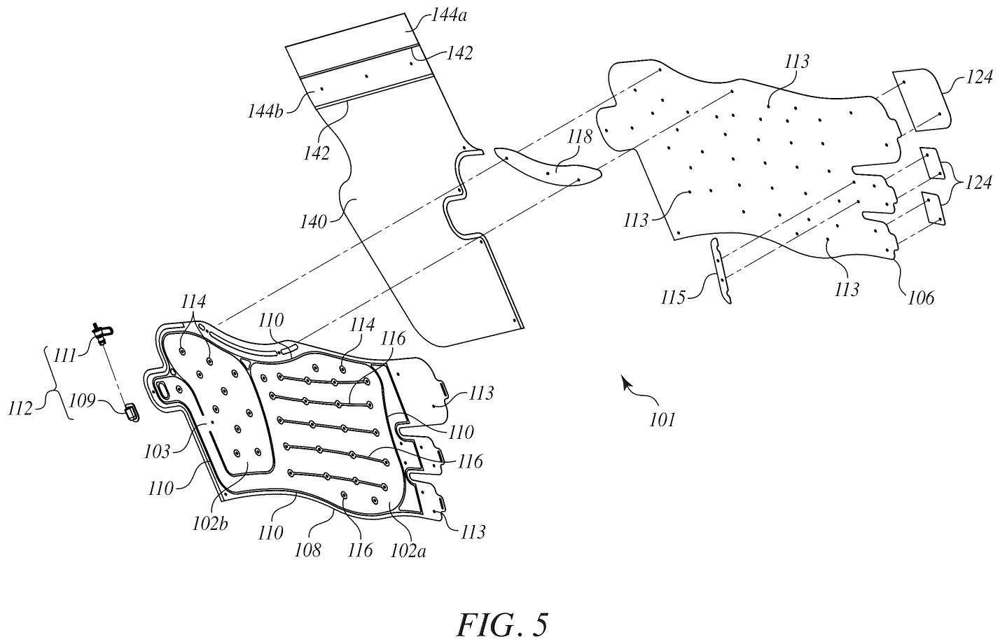

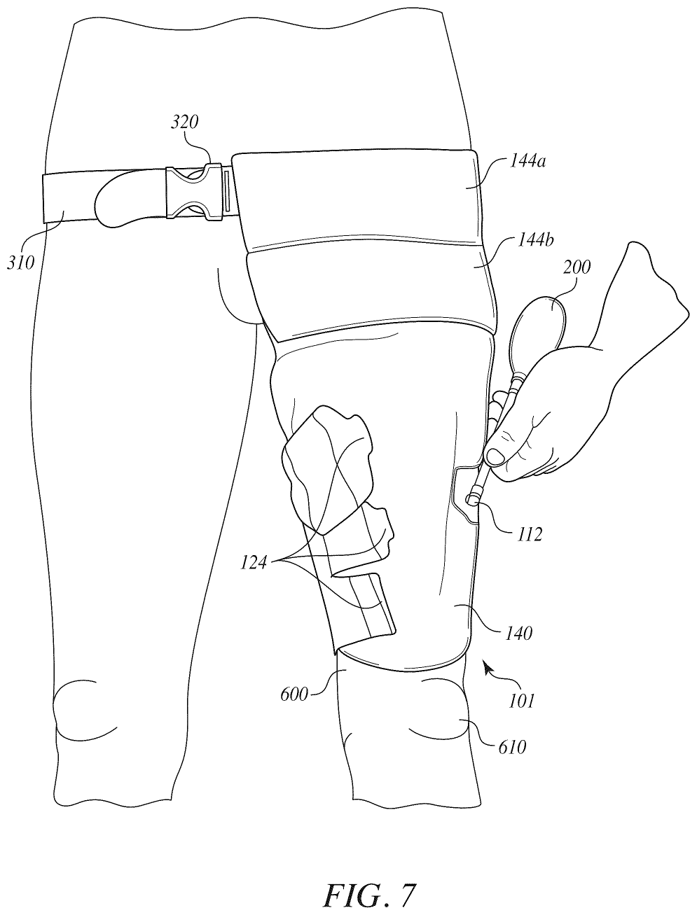

Further, the inventive saphenous vein compression system includes a thigh therapeutic compression garment, As shown in FIGS. 4-7 the thigh therapeutic compression garment is an inflatable compression wrap 101 placed over, in this embodiment, an elastic compression sleeve 160 or could also be a compression stocking or any other elastic material. Other combinations may be employed as long as a compression garment 101 is placed over the bolster dressing 150 and over the elastic compression sleeve 160.

In one embodiment as shown in FIGS. 4-7 the thigh therapeutic compression garment is an inflatable thigh therapeutic compression apparatus 101 including a compression bladder 102 includes a medial bladder portion 102A and a lateral bladder portion 102B. The medial bladder portion 102A when in use is located along the medial saphenous vein of the individual on the inner thigh and the lateral bladder portion 102b when in use is located along the lateral saphenous vein of the individual on the back thigh. The thigh therapeutic compression apparatus 101 is configured and adapted to wrap around a patient's limb such as in this instance a left thigh, though it could be used in other limbs. An embodiment not shown of the saphenous vein compression system 100 including a thigh compression apparatus 101 which was configured and adapted to wrap around a patient's limb such as in this instance a right thigh would be the mirror image of the left thigh compression apparatus 101 as shown in FIGS. 4-7 so that the medial bladder 102A would be located on the inner thigh arca of the right leg and the lateral bladder portion 102B (if included) would be located on the back thigh of the user. The thigh therapeutic compression apparatus 101 is formed out of continuous outer sheet and inner sheet forming a bladder within and has proximal end portion (top as oriented in FIG. 4-7) and opposed distal end portion (bottom as oriented in FIGS. 4-7) which is configured and adapted to conform around a patient's thigh and provide compression through the inflation of bladder 102. Inner sheet and outer sheet are made out of a nylon laminated polyurethane sheet which are configured and adapted to be RF welded together. However, any other suitable materials which are weldable or otherwise joined while being airtight can be used. Continuous peripheral weld line forms an airtight boundary of integrally formed bladder 102. In this exemplary embodiment, bladder 102 is a single continuous bladder throughout however, it is envisioned that the thigh therapeutic compression apparatus 101 could have an independent bladder either separately inflatable or inflatable through a one-way valve or other desired inflation/deflation configuration.

In this embodiment, hook and loop fasteners 124 are provided along the edge of inner and outer sheets in order to ease adjustment and secure therapeutic compression apparatus 101 on a patient's limb such as for example a thigh. It is envisioned that the therapeutic compression apparatus 101 can also be secured to a patient's thigh by other means, such as zippered, buttoned, or be cuff shaped by other such suitable means. Further, it is also envisioned that hook and loop closures 124 can be replaced by material similar to that of stabilization means 140 (such as a hip or waist stabilizer) described below and be welded/sewn/attached to bladder 102 for improved comfort. In this embodiment the thigh therapeutic compression apparatus 101 is inflatable via an inflation port 112 connected to an inflation means such as device which is a hand pump (not shown) capable of attaching to inflation port 112 to inflate bladder 102 (102A, 102B). It can be appreciated that a mechanical or automatic inflation pump (not shown) can also be attached to inflation port 112 to inflate and deflate bladder 102 (102A, 102B) to provide pulsating pressure to a user's thigh. A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. A vent valve (not shown) can also be incorporated into the thigh therapeutic compression garment 101 or with inflation means to allow a user to selectively deflate bladder 102 (102A, 102B). Further, a check valve or relief valve is incorporated with either inflation means or bladder I 02 (102A, 102B) to prevent over-inflation once a maximum pressure is detected. Examples of relief valves are described in U.S. Pat. No. 7,276,037 and U.S. Pat. No. 7,850,629, the disclosures of which are incorporated by reference in their entirety. In this embodiment a panel 118 is also included located along the proximal end portion of the saphenous vein compression system configured to conform to the groin area of the leg. The panel 118 may be comprised of a flexible material such as but not limited to nylon, polyurethane, cotton, or other suitable material, or a material such as Spandex. The panel 118 may be configured to prevent the occurrence of a pinch point and reduce pressure on a patient's groin area when wearing the thigh therapeutic compression garment 101.

Within the thigh therapeutic compression garment 101 is a number of spot welds chosen and located to create a desired preconfigured compression gradient profile which can be obtained cost-effectively. A number of different embodiments of bladder configurations can be used in thigh therapeutic compression garment 101 of the subject invention such as those configurations described above. In one embodiment as shown the thigh therapeutic compression garment 101 has fluid chambers, the bladder 102 comprised of two bladders with air flowing between each bladder, for example the medial bladder 102A located in use along the inner portion of the patient's thigh and the lateral bladder 102B located in use along the patient's back and/or outer thigh. In this exemplary embodiment, bladder 102 is a single continuous bladder comprised of two portions of a bladder such as the medial bladder 102A and lateral bladder 102B. This configuration is particularly useful when compression is needed to improve fluid movement (e.g., blood, lymph, etc.) within the body. Further, linear weld lines 116 allow for better compression along the inner thigh and outer thigh of a patient's thigh by increasing tension applied to the medial portion of the thigh of a patient and thus further expanding the predetermined gradient compression profile. This increased tension can generate a more effective thigh compression in order to increase venous flow. Linear weld lines 116 located horizontally along the medial portion of the thigh create a ribbed portion, which keeps the inflated profile of therapeutic compression apparatus 101 compact which can further increase ambulation and reduce interference with a patient's clothes. While not shown, the linear weld lines 116 could also be located vertically along the medial bladder portion 102A and/or the lateral bladder portion 102B, such as vertical linear weld lines. Other combinations of linear weld lines and spot welds may be used to create a predetermined gradient compression profile for the bladders 102, 102A, 102B, 402 and thus apply gradient compression onto the leg or other limb, body part when in use, some of which are included by way of example in U.S. application Ser. No. 17/608,323 and 16/846,211.

It can be appreciated that depending on the location of the saphenous vein compression system, different pressure gradients or compression gradients may be utilized. Other examples of other bladder pressure gradient profiles are described herein. Other possible gradient pressure profiles or compression gradient profiles may be imagined based upon the geometric location of the spot welds alone or in connection with the linear welds. Once the thigh therapeutic compression garment 101 is secured around a patient's thigh, bladder 102, 102A, 102B is inflated and not able to shift out of place, thus increasing comfort and reducing fitting issues on the patient. In order to increase the ease of ambulation by a patient, in this exemplary embodiment, the proximal portion (upper edge located closer to the hip and pelvis) of the thigh therapeutic compression garment 101 includes a securing means, which is shown in FIGS. 4-7 as a stabilizer 140 so as to stabilize and secure the thigh therapeutic compression garment 101 to the thigh of the user and reduce slippage of the thigh therapeutic compression garment 101 down towards the knee. Any stabilizing or securing means may be employed such as for instance, and not show, a hip or waist strap can be used. If a strap is used, the strap may be pulled tight against a patient's leg and hip so as to reduce slippage of the thigh therapeutic compression garment 101 down the knee and may also improve compression near the hip area of a patient. In this embodiment, the stabilizing means or securing means 140 is comprised of a material that is comfortable to the user, and possibly thin so as to worn either under pants or over pants. The stabilizing mean or securing means 140 is connected to the wrap along the side closest to the lateral bladder portion I 02B and from the proximal end to the distal end of the wrap. The stabilizing means or securing means 140 is comprised of cotton, doe skin, leather, rubber, Spandex or any other material. The stabilizing means or securing means 140 may include a panel 140 and at least two apertures 144A, 144B on the proximal end configured wherein a belt 300 or other securing means can be inserted and then secured around the waist of the individual when in use. In this embodiment of the thigh therapeutic compression garment 101, the securing means is a panel 140 which includes at least one aperture 144 or two 144A, 144B, created by a linear 142 weld(s) or other sealing means such as a linear stitch, linear adhesion, linear fold, or the like. The apertures 144A, 144B are configured, in this embodiment, to allow a belt system 300 to be inserted within one of the apertures 144A, 144B so as to secure the thigh therapeutic compression garment 101 to the waist of a patient and to reduce slippage of the thigh therapeutic compression garment 101 downward towards the knee. While only one aperture 144 may be employed (not shown), the configuration including at least two apertures 144A, 144B is suggested so that each user can adjust the placement of the thigh therapeutic compression garment 101 along the user's thigh given the user's height, length of leg, waist, and the like. Other configurations may also be employed having three or more apertures 144. The panel 140 may be comprised of a flexible material such as but not limited to nylon, polyurethane, cotton, or other suitable material. or a material such as Spandex. The panel 140 may be connected to the thigh therapeutic compression garment 101 by a RF weld, adhesive, glue or any other known connecting means.

An embodiment of the saphenous vein compression system 100 may further include (not shown) another second or third or multiples of absorbent foam, sponge or dressing to be used alone or with a suction conduit coupled to a suction apparatus, and may further include wound dressing impregnated with medication, pain relief, silver, zinc oxide or other materials known to improve healing or reduce infection and/or pain at the wound site. Other embodiments of the compression mechanism are possible such as a combination of a sealed fluid-filled bladder, an absorbent foam, sponge or dressing, and a suction conduit for use with venous ulcers or other open wounds. The absorbent foam, sponge or dressing (not shown and hereinafter referred to as “the sponge”) may have the ability to absorb exudate from the wound or ulcer. In addition, the optional sponge preferably has an open-cell structure which aids in wicking the exudate from the wound or ulcer. The optional sponge may be lightly affixed to the patient's skin by an adhesive film such that the optional sponge may be peeled off and replaced. Some embodiments may include a suction conduit for example provided in between the saphenous vein compression system 100 and the optional sponge. More particularly, the suction conduit may be oriented downwardly towards the sponge so that when a source of negative pressure is applied to the suction conduit, that negative pressure is applied to the wound or ulcer via the sponge. As a result, exudate can be wicked and suctioned away from the wound or ulcer via the sponge and into the conduit (such as a tube and other devices). In one embodiment, the conduit may be easily detached from the saphenous vein compression system 100 so that the patient can walk or be transported away from the source of suction while pressure is still being applied to the wound or ulcer. In another embodiment not shown the bolster dressing 150 itself may include a suction conduit to suction away exudate from the wound location. Other accessories or other bandages, sponges and the like may be employed with the saphenous vein compression system 100.

It can be appreciated that depending on the location of the therapeutic compression apparatus being placed on the patient's body part or limb, different compression gradient profiles may be utilized. Further, different pressure gradients may be employed depending on the treatment (general swelling, lactic acid build up, lymphedema, post-sclerotherapy, CVT, DVT, etc.) and the treatment site. Other examples of bladder compression gradient profiles are described in U.S. patent application Ser. No. 12/911,563 and U.S. patent application Ser. No. 12/855,185, and U.S. Ser. No. 16/846,211 and U.S. Ser. No. 17/608,323, the disclosures of which are incorporated by reference in their entirety.

A number or variety of inflation means can be employed such as a manual pump, hand pump, foot pump, mechanical pump, electrical pump, battery-operated pump, static pump, intermittent pump, varying pump, automatic pump, pneumatic pump, negative pressure pump, suction pump or vacuum, pulsing pump, or any other known or developed source of inflation so as to provide a certain pressure within the bladder so to provide compression in use by the patient. Further, the inflation means could include a means to monitor or regulate the inflation. The inflation means could include programming such that the bladder 102, 102A, 102B is inflated and deflated to a set pressure at intervals or at set times throughout the day or night when the compression apparatus is in use worn on the patient. For instance, by way of example only, the inflation means could be set to 40 mm-Hg at 9 am and then set to deflate to 20 mm-Hg at 11 am and then set to inflate to 30 mm-Hg at 12 pm and so on throughout the day and night for each patient individually. In another embodiment of the present invention, if the lateral bladder 102B is sealed and separate from the medial bladder 102A, then there may be two separate inflation ports 112 (e.g. 112 A and 112B) that are each connected to either the same or different inflation means, and the pressure levels of the medial bladder 102A and the lateral bladder 102B could be the same or have different pressure levels. For instance, by way of example only, the inflation means could be set to 40 mm-Hg for the medial bladder and set to 20 mm-Hg for the lateral bladder, or each could vary and the inflation means be set to inflate, deflate, inflate, etc. throughout the day as described above.

It is envisioned, that the inventive saphenous vein compression system 100 could be conjunction with a lower leg compression apparatus such as shown co-owned U.S. Pat. No. 9,033,906 and U.S. Pat. No. 7,967,766 and U.S. Pat. No. 7,559,908 and U.S. Pat. No. 7,276,037 and Ser. No. 12/911,563 and Ser. No. 12/855,185, and Ser. No. 14/714,706 and Ser. No. 16/846,211 and U.S. Ser. No. 17/608,323. Further, one inflation means could be connected to each of the two compression apparatus. The inflation means could include programming such that the bladder of both the saphenous vein compression system and the bladder or bladders of the lower leg compression apparatus are each (or together at the same time) inflated and deflated to a set pressure at intervals or at set times throughout the day or night when the compression apparatus is in use worn on the patient. For instance, by way of example only, the inflation means could be set to 40 mm-Hg at 9 am and then set to deflate to 20 mm-Hg at 11 am and then set to inflate to 30 mm-Hg at a 2 pm and so on throughout the day and night for each patient individually. In another embodiment present invention, for instance, by way of example only, the inflation means could be set to 40 mm-HG for the thigh bladder and set to 20 mm-Hg for the lower leg bladder (or calf bladder or foot bladder, etc.), and could vary with the inflation means be set to inflate, deflate, inflate, etc. throughout the day as described above. Depending on the treatment plan for the patient and depending on the specific patient, each of them could be set to the same or different pressure levels throughout the day and night.

Post-operative sclerotherapy treatment can also be effective with the saphenous vein compression system 100 including a thigh therapeutic compression apparatus 101 wherein the lateral bladder 102B is a closing means (not shown) over the gap 103 and either remains in a non-inflated state or may inflated to a lower pressure level, or could be inflated to any pressure level desire by the patient. Typically varicose veins are located on the inner thighs of an individual patient and for post-operative sclerotherapy treatment compression is needed on the surgical site along the inner thigh. The embodiment of the inventive saphenous vein compression system 100 as shown can be used for specific compression only along the inner thigh of the patient's leg, such as post-operative sclerotherapy procedures. Other post-operative procedures may employ the same method. Other treatments such as lymphedema may employ the bolster dressing 150 with an elastic or compression dressing or garment 160, and the therapeutic compression garment 101 in an inflated state so as to apply compression and pressure to the selected site on the patient's thigh.

In use the bolster dressing 150 is activated by the user removing the cover layer 155 exposing the adhesive layer 154 and adhering the bolster dressing 150 to the skin located along the saphenous vein. Then the elastic or compression sleeve or liner or housing or sticking 160 is placed over the bolster dressing 150 so as to apply compression on the entire thigh or even leg with additional localized compression or pressure over the bolster dressing 150. The thigh therapeutic compression garment 101 is placed or applied over both the bolster dressing 150 and elastic liner or stocking 160 and then secured around a patient's thigh via the securing means such as the belt system 310, 320. Once bladder 102 (102A, 102B) is inflated and not able to shift out of place, there is compression applied to the thigh area and specifically along the saphenous vein resulting in increased comfort to the patient and reducing fitting issues on the patient most notably slippage of a thigh compression wrap when walking by the patient. In order to increase the case of ambulation by a patient, in this exemplary embodiment, the proximal portion (upper edge located closer to the hip and pelvis) of the thigh therapeutic compression garment 101 includes a securing means, so as to secure the thigh therapeutic compression garment 101 to the thigh of the user and reduce slippage of the thigh therapeutic compression garment 101 down towards the knee and the further secure the bolster dressing 150 to the skin of the patient. Any securing means may be employed such as for instance, and not show, a hip or waist strap can be used. If a strap is used, the strap may be pulled tight against a patient's leg and hip so as to reduce slippage of the thigh therapeutic compression garment 101 down the knee and may also improve compression near the hip area of a patient.

Another embodiment of the saphenous vein compression system 100 not shown may further include a second or multiples of absorbent foam, sponge or dressing to be used alone or with a suction conduit coupled to a suction apparatus as disclosed above. Other embodiments of the compression mechanism are possible such as a combination of a sealed fluid-filled bladder, an absorbent foam, sponge or dressing, and a suction conduit for use with venous ulcers or other open wounds. The second or multiples optional absorbent foam, sponge or dressing (not shown and hereinafter referred to as “the sponge”) with the suction conduit may have the ability to absorb exudate from the wound or ulcer. In addition, this second or multiples optional sponge preferably has an open-cell structure which aids in wicking the exudate from the wound or ulcer. The second or multiples optional sponge may be lightly affixed to the patient's skin by an adhesive film such that the sponge may be peeled off and replaced. Some embodiments may include a suction conduit, for example provided in between the saphenous vein compression system 100 and the second or multiples optional sponge. More particularly. the suction conduit may be oriented downwardly towards the sponge so that when a source of negative pressure is applied to the suction conduit, that negative pressure is applied to the wound or ulcer via the second or multiples optional sponge. As a result, exudate can be wicked and suctioned away from the wound or ulcer via the second or multiples optional sponge and into the conduit (such as a tube and other devices). In one embodiment, the conduit may be easily detached from the saphenous vein compression system 100 so that the patient can walk or be transported away from the source of suction while pressure is still being applied to the wound or ulcer.

The inventive saphenous vein compression system 100 may be included in a kit having various wound dressings and/or bandages. The wound dressings and/or bandages may be disposed of on a more frequent basis and the inventive therapeutic compression apparatus is applied in conjunction or combination with the wound dressings and/or bandages. In one embodiment the therapeutic compression apparatus is used over or on top of the wound dressing applied to the skin. Other components of the kit may include bandages, gauze or other wound dressings impregnated with medication, pain relief, silver, zinc oxide or other materials known to improve healing or reduce infection and/or pain at the wound site.

The present invention has been illustrated and described with respect to specific embodiments thereof, which embodiments are exemplary and illustrative of the principles of the invention and are not intended to be exclusive or otherwise limiting embodiments. For instance, while in the foregoing embodiments the therapeutic compression apparatus are described as having inflatable bladders, the therapeutic compression apparatus may additionally include integrally formed or attached (e.g., by adhesive, radio-frequency welding, etc.) compression members that are not configured for inflation and/or deflation. For instance, additional compression members may be implemented using any of a variety of preformed and/or prefilled cushioning materials such as foam cushions and/or air, gel, or other fluid filled non-inflatable cushions, provided such compression members generate sufficient compression in combination with integral compression bladders. Further, while particular shapes, sizes, and materials have been described for purposes of illustration, it will be recognized that any of a variety of shapes or sizes can be used, and the materials described are not exclusive but merely illustrative. Also, as noted above, while the bladder shown is inflated with air, it will be appreciated that any other fluid or medium such as liquid or gel can be used. Moreover, as also noted, it will be understood that bladders may be configured to have multiple pneumatically independent and/or pneumatically coupled bladder sections, and may also be configured to have various contours or lobulations.

The saphenous vein compression system 100 described herein can be used for any suitable condition treatable by compression therapy and the like. For example, therapeutic compression apparatus in accordance with the present invention can be used for compression of the venous system for the treatment of venous ulcers, CVI, DVT, for the treatment of lymphedema (where it is circulation of fluids in the lymph system rather than in the venous system that is promoted) and the like. The inventive saphenous vein compression system may also optionally include various dressings such as but not limited to bandages, gauze, cotton, foams, lints, plasters, and synthetic wound dressings which includes hydrogels, hydrocolloids, alginates, synthetic foam dressing, silicone meshes, tissue adhesives, vapor-permeable adhesive films and silver/collagen containing dressing. Optional heat may be applied under or within the thigh therapeutic compression garment.

The saphenous vein compression system of the instant invention described herein solves many problems with the prior art and in the industry and treatment of patients. The thigh therapeutic compression apparatus may be applied on the patient's body part by the patient without the need or requirement of a skilled caregiver as required by current devices and apparatus. It further is capable of maintaining sufficient effective pressure without overpressure complications, maintaining compression and the like.

The saphenous vein compression system of the instant invention also reduces the problem is leakage of set compression within the treatment apparatus and devices, bandages, stockings and hosiery and instead promotes a more effective treatment for CVI, DVT and/or lymphedema and other treatments. The saphenous vein compression system of the instant invention also reduces the problem of slippage downward towards the knee of the patient when the apparatus is in use, especially when the patient is walking or moving about, which slippage is a major deterrent by patients in using current compression systems along the thigh area and saphenous vein area. Patients complain of slippage down the leg and end up removing the known compression systems which then impedes recovery and healing and leads to increased complications, infection and the like. The inventive saphenous vein compression system solves this problem and in contrast encourages the user to keep the inventive saphenous vein compression system on during the day when moving around thereby aiding in healing and pain management and reducing complications, infection, pain and the like. Further, the user themselves can remove and reapply the inventive saphenous vein compression system without the need for a caregiver or medical professional, again thereby increasing use by the patient and effectiveness of the inventive compression treatment and method of use.

The present invention may assist in treatments wherein compression on the long saphenous vein may be recommended in treatment of certain conditions. Other embodiments of the present invention may assist in treatments for other conditions there may be less compression recommendation on the long saphenous vein on the medial side of the leg and more compression recommended on the short saphenous vein of the leg which runs on the back portion of the leg. including the thigh. In other embodiments of the present invention with only one lateral bladder and no medial bladder, the inventive therapeutic compression apparatus may assist in treatment of conditions where compression is recommended only on the short saphenous vein of the leg. These and other needs are met by the inventive saphenous vein compression system 100.

Another embodiment (not shown) may be a full leg compression apparatus including a thigh bladder a calf bladder, an optional foot bladder, and a knee opening (or an optional knee pad) and an optional heel opening and foot strap. In this configuration. the thigh bladder may extend below the knee joint level and apply compression around the patella of the knee area. It may also have suspender hooks or slots (not shown) that allow for suspenders to be mated thereto in order to support the apparatus by a waist band when in use. The suspenders limit downward travel of the apparatus when in use such that it maintains its desired position.

Thus the inventive saphenous vein compression system solves many problems with current known apparatus, garments, systems and methods of use. The inventive saphenous vein compression system solves the issues with wound dressing for venous disease treatments and improves and standardizes localized bolstering. The combination of the bolster dressing with the elastic sleeve and thigh therapeutic compression garment provides a standardized bolster height as well as the ability to distribute the pressure evenly across the underlying anatomy resulting in optimal compression and comfort to the user and reducing pressure ulcers while at the same time assisting in healing and reducing pain and risk of complications and infection compared to current standard apparatus and treatment of methods of use.

The inventive saphenous vein compression system combines a bolster dressing with at least two layers and levels of thigh compression resulting in localized compression post-ablation on saphenous vein. Studies show that the inventive saphenous vein compression system improves pressure and compression on the saphenous vein resulting in increased comfort for patient and a importantly reduced slippage of the compression system down leg. Further, the inventive saphenous vein compression system elevates the wound area via the bolster dressing which also exudates the wound area resulting in improved healing and reduced pressure ulcers, complications, infections and the like. The patients and users have increased compliance with their treatment plan and therefore improved healing with less complications.

While the subject invention of the present disclosure has been described with respect to preferred and exemplary embodiments, those skilled in the art will readily appreciate that various changes and/or modifications can be made to the invention without departing from the spirit or scope of the invention as described herein. There have been described and illustrated herein several embodiments of an intermittent pressure apparatus and a method of installing and operating the same. While particular embodiments of the invention have been described, it is not intended that the invention be limited thereto. as it is intended that the invention be as broad in scope as the art will allow and that the specification be read likewise. Thus, while particular shapes and sizes of inflatable bladders and straps have been disclosed. it will be appreciated that other shapes, sizes. and attachment means may be used as well. It will also be understood that while Velcro and adhesive means have been disclosed for helping to secure the bladders to the leg and foot. other types of attachments such as hooks, snaps. or wraps may be used. In addition, it will be appreciated that while the fluid conduit may be detachably connected to the bladders using mating threaded portions or bayonet locks, other means of attachment known in the art may be used. It will therefore be appreciated by those skilled in the art that yet other modifications could be made to the provided invention without deviating from its spirit and scope as claimed.

We claim:

1. A saphenous vein compression system for use on a thigh of a user, comprising:
a wound bolster comprised of an absorbent material, an adhesive, and a pre-filled bladder;
a compression tubing; and
a thigh therapeutic compression garment including (a) at least one bladder configured to be located along the medial area of the thigh and the at least one bladder having a number of welds forming predetermined compression gradient along the saphenous vein of the user when inflated, (b) a first means for locating the at least one bladder along the medial area of the thigh above a knee of the human body when in use, (c) a second means for locating the wrap along a groin and hip area of the human body when in use, (d) an inflation means connected to the at least one bladder via a universal inflation port wherein the inflation means includes a check valve to protect from over inflation and set to open at a predetermined pressure or a user selectable pressure, and wherein the universal inflation port is configured to be universally capable of connection to both a static and/or an intermittent inflation means to provide static and/or intermittent compression to a body part of a user when the at least one bladder is inflated by the inflation means with the single inflation port including a check valve as part of the closed system configured so that when the inflation means is disconnected the check valve closes and maintains the set pressure within the at least one air bladder.

2. The saphenous vein compression system of claim 1 wherein the thigh therapeutic compression garment includes (a) an inner sheet configured and adapted to be disposed facing the thigh when in use and (b) an outer sheet joined to the inner sheet with the at least one bladder defined between the inner and outer sheets.

3. The saphenous vein compression system of claim 1 wherein the inflation means is selected from the group consisting of manual pumps, static pumps, intermittent pumps, electrical inflation pumps, battery inflation pumps, gas powered inflation pumps, static pneumatic compression pumps, intermittent pneumatic pressure pumps, and the combination thereof.

4. The saphenous vein compression system of claim 1 wherein the first means for locating is a first attachment member wrapping around the thigh to secure the wrap to the thigh of the user.

5. The saphenous vein compression system of claim 4 wherein the first attachment member is a plurality of straps attached to the thigh therapeutic compression garment.

6. The saphenous vein compression system of claim 1, wherein the second means for locating is a second attachment member wrapping around the groin and hip and attached to a securing means around a waist of the human body when in use.

7. The saphenous vein compression system of claim 6 wherein the second attachment member is a panel attached to the thigh therapeutic compression garment and extending above a proximal end of the wrap, including at least one aperture on a proximal end of the panel configured to be capable of insertion of the securing means around the waist of the user.

8. The saphenous vein compression system of claim 6, wherein the securing means includes a belt or straps for securing around the waist.

9. The saphenous vein compression system of claim 1, wherein the at least one bladder has at least two chambers configured wherein one chamber is located along the medial area of the thigh and a second chamber is located along a lateral area of the thigh.

10. The saphenous vein compression system of claim 9, wherein the at least two chambers of the at least one bladder are configured such that when the fluid is forced out of one chamber it applies an extra pressure to the other chamber in the range of about 10 mm Hg and 20 mm Hg extra pressure, and wherein the predetermined compression profiles are different in each chamber.

11. The saphenous vein compression system of claim 9, wherein the at least two chambers are not in fluid connection and may have different predetermined compression profiles when inflated, wherein the second chamber has a second universal inflation port.

12. The saphenous vein compression system of claim 1 further comprising an elbow, a check valve and a valve cap wherein the valve cap is capable of releasing a pressure created by the inflation means within the at least one bladder.

13. The saphenous vein compression system of claim 1 wherein the inflation means includes a three-way switch configured to be capable of switching among a first valve, a second valve and a third valve with the first valve configured to be capable of holding a pressure created by the inflation means within the at least one bladder creating an inflated state of the at least one bladder, the second valve configured to be capable of releasing a set amount of pressure within the at least one bladder creating a partially deflated state of the at least one bladder, and the third valve configured to be capable of releasing all the pressure within the at least one bladder so as to create a fully deflated state of the at least one bladder.

14. A method for applying compression to a thigh of a human body along the saphenous vein, comprising:
removing a protective layer to expose an adhesive layer on a wound bolster, the wound bolster including an absorbent layer above the adhesive layer and a pre-filled bladder above the absorbent layer;
adhering the wound bolster along the saphenous vein on the thigh of a user via the adhesive layer;
placing a compression liner over the wound bolster on the thigh of the user;
affixing a thigh therapeutic compression garment to the thigh of the user, wherein the thigh therapeutic compression garment includes (a) at least one air bladder connected to an inflation means and wherein the at least one air bladder has a plurality of welds configured to create a predetermined compression profile when inflated and (b) an inflation port having a face connector, a check valve and a valve cap wherein the valve cap is capable of releasing a pressure created by the inflation means within the at least one air bladder;
securing the thigh therapeutic compression garment to the thigh with a securing means attached to the thigh therapeutic compression garment so that the at least one air bladder is located in the medial area of the thigh over the saphenous vein of the user;
stabilizing the thigh therapeutic compression garment along a waist or hip using a stabilizing means;
inflating the at least one air bladder in the thigh therapeutic compression garment;
closing the valve cap on the universal inflation port on the thigh therapeutic compression garment; and
walking such that the thigh therapeutic compression garment is secure and stabilize on the thigh of the user while applying compression to the saphenous vein of the user's thigh.

15. The method of claim 14, wherein the at least one air bladder is configured to have one chamber capable of sequential gradient pressure when connected to the inflation means.

16. The method of claim 14, wherein the inflation means includes a three-way switch configured to be capable of holding a pressure created by the inflation means within the at least one bladder creating an inflated state of the bladder, capable of releasing a set amount of pressure within the at least one bladder creating a partially deflated state of the bladder, and capable of releasing all the pressure within the at least one bladder so as to create a fully deflated state of the at least one bladder.

17. The method of claim 14, wherein the at least one bladder has at least two chambers configured wherein one chamber is located along the medial area of the thigh and a second chamber is located along a lateral area of the thigh, with the at least two chambers either in fluid connection with one another or separately inflatable.

18. A saphenous vein compression system for the thigh of a user, comprising:
a wound bolster comprised of an absorbent material, an adhesive, and a pre-filled bladder;
a compression tubing; and
a thigh therapeutic compression garment including (a) at least one bladder configured to be located along the medial area of the thigh and the at least one bladder having a number of welds forming predetermined compression gradient along the saphenous vein of the user when inflated, (b) a first means for locating the at least one bladder along the medial area of the thigh above a knee of the human body when in use, (c) a second means for locating the wrap along a groin and hip area of the human body when in use, (d) an inflation means connected to the at least one bladder via a universal inflation port wherein the inflation means includes a check valve to protect from over inflation and set to open at a predetermined pressure or a user selectable pressure, and wherein the universal inflation port is configured to be universally capable of connection to both a static and/or an intermittent inflation means to provide static and/or intermittent compression to a body part of a user when the at least one bladder is inflated by the inflation means with the single inflation port including a check valve as part of the closed system configured so that when the inflation means is disconnected the check valve closes and maintains the set pressure within the at least one air bladder; and
an assembly comprising: (1) a pressure mechanism having a flexible member for attachment around a limb and an air chamber which assumes a first depressurized state and a second pressurized state, said air chamber having a length and a width, said width being less than half the width of the flexible member; (2) a pre-filled air bladder having a length and a width smaller than the width of the air chamber; (3) an absorbent foam, sponge or dressing coupled to the pre-filled air bladder; and (4) a suction conduit in fluid communication with the absorbent foam, sponge or dressing and adapted for coupling to a source of negative pressure.

19. The saphenous vein compression system of claim 18 wherein the assembly suction conduit terminates on one end in a fluid connector and the assembly pre-filled air bladder defines a central opening, and the suction conduit extends into the central opening.
